(19) United States
(12) Patent Application Publication
(10) Pub. No.: US 2025/0031838 A1
(43) Pub. Date: Jan. 30, 2025

(54) APPARATUS AND HAIRBRUSH FOR ASSESSING HAIR CHARACTERISTICS

(71) Applicant: BASF SE, Ludwigshafen (DE)

(72) Inventors: Zuebeyde Dogan, Siegen (DE); Jun Tao Xia, Shanghai (CN); Juergen Falkowski, Düsseldorf-Holthausen (DE); Sascha Hoeche, Düsseldorf-Holthausen (DE); Christina Kohlmann, Monheim (DE); JunMin Lee, Suwon-si (KR); Wei Hsiang Lin, Suwon-si (KR)

(21) Appl. No.: 18/715,784

(22) PCT Filed: Dec. 6, 2022
(86) PCT No.: PCT/EP2022/084663

§ 371 Date: Jun. 3, 2024

(30) Foreign Application Priority Data
Dec. 8, 2021 (WO) PCT/CN2021/136335

(51) Int. Cl. A46B 15/00; A46B 9/02; A61B 5/00

(52) U.S. Cl. CPC A46B 15/0006; A46B 9/023; A46B 9/026; A61B 5/448; A46B 2200/104

(57) ABSTRACT

The invention refers to an apparatus (120) for accessing hair characteristic while brushing hair of a subject, wherein the apparatus comprises a vibration measurement providing unit (121) for providing a vibration signal indicative of a result of a vibration measurement performed by one or more vibration sensors (112) integrated into a hairbrush (110) comprising a plurality of bristles (111). The vibration measurements are performed by the vibration sensors during a brushing of the hair using the hairbrush and are indicative of vibrations of the bristles of the hairbrush during the brushing. A hair characteristic determination unit (122) then determines a hair characteristic based on the vibration signal.

### FIELD OF THE INVENTION

The invention relates to an apparatus, a hairbrush, a hairbrush system comprising the apparatus and the hairbrush, a method and a computer program product for assessing hair characteristics by brushing hair of a subject.

### BACKGROUND OF THE INVENTION

Generally, there is a growing trend to use care products with additional functionalities, in particular, evaluation functionalities. Devices with such additional functionalities, i.e., functionalities going beyond the originally intended functionalities, are often called “smart” devices. Some of these smart devices already allow to track an individual hair and/or scalp condition and/or improvement. However, the existing devices are often not reliable or efficient. Moreover, home useable devices should also be self-explaining such that they can directly and easily be used by the end consumer.

Presently available devices for hair analysis are in many cases explicitly designed for a usage by hairdressers. Other available devices for home usage use imaging techniques for the hair analysis that is often difficult to apply and, if not applied correctly, leads to unreliable results in addition to being a quite costly technology.

Document WO 2021/237236 A1 discloses a hair grooming device, system and method for measuring interaction with hair of a person during a hair grooming event, including a processor readable storage medium, storing executable instructions and a processor in communication with the processor readable storage medium.

It would however be advantageous if a home, hair salon, or test institute usable device could be provided to a user that allows for a reliable and accurate hair evaluation and is further easily and directly applicable by a user.

### SUMMARY OF THE INVENTION

It is an object of the present invention to provide an apparatus, a hairbrush, a system comprising the apparatus and the hairbrush, a method and a computer program product that allow for a reliable and accurate hair evaluation that can be easily utilized by a user in his/her everyday routine.

In a first aspect of the present invention, an apparatus for accessing hair characteristic while brushing hair of a subject is presented, wherein the apparatus comprises a) a vibration measurement providing unit for providing a vibration signal indicative of a result of a vibration measurement performed by one or more vibration sensors integrated into a hairbrush comprising a plurality of bristles, wherein the vibration measurements are performed by the vibration sensors during a brushing of the hair using the hairbrush and are indicative of vibrations of the bristles of the hairbrush during the brushing, and b) a hair characteristic determination unit for determining a hair characteristic based on the vibration signal.

The inventors have found that different hair characteristics like hair damage, hair thickness, or hair type, lead to different vibrations of the bristles during a brushing of the hair. Thus, by utilizing vibration measurements performed by one or more vibration sensors integrated into a hairbrush that are indicative of the vibrations of the bristles of the hairbrush during the brushing, hair characteristics can be determined based on the vibration signal very reliably and accurately. Moreover, since the measurements are performed during the normal brushing of the hair, which is a normal everyday routine, it is very easy for a user to provide these measurements and thus to utilize the apparatus. Hence, the apparatus and also the system comprising the apparatus and a respective hairbrush that can provide the measurements allows for a reliable and accurate determination of the hair condition and are further easily usable in an everyday routine of a user.

Generally, the apparatus can be realized in form of any hard- and/or software provided on any kind of computer device. Moreover, the functions provided by the units of the apparatus can also be distributed over more than one general computing device, for instance, can be performed by a network or in a cloud computing. Preferably, the functions provided by the apparatus are realized as part of the soft- and/or hardware of a computational user device like a smartphone, a tablet, etc. However, the apparatus can also be realized as a dedicated standalone device, for instance, being part of a hairbrush providing the vibration measurements and can then be provided, for instance, in a handle of the hairbrush and can be adapted to be communicatively coupled to an output device for outputting the result of the hair characteristic determination to a user. Such an output device can then again refer to a user computational device like a smartphone, tablet, laptop, etc., but can also be provided as part of the hairbrush, for instance, in the form of a small display device.

Generally, the subject can refer to a living being or an inanimate object comprising hair. In case of a living being, it is preferred that the subject refers to a human being, however, the subject can also refer to an animal like a dog or a cat. In case of an inanimate object, the subject can refer to a hair strand, wig or any other form of hair replacement.

The vibration measurement providing unit is adapted to provide a vibration signal. Generally, the vibration measurement providing unit can refer to or can be communicatively coupled to a storage unit on which the vibration signal is already stored. However, the vibration measurement providing unit can also be in direct communication with the one or more vibration sensors providing the vibration signal and can then directly provide the vibration signal. Moreover, the vibration measurement providing unit can also be regarded as a receiving unit for receiving the vibration signal, for instance, from a long- or short-term storage on which the vibration signal is stored or directly from the one or more vibration sensors.

The vibration signal is indicative of a result of a vibration measurement performed by one or more vibration sensors. The vibration sensors performing the vibration measurement are integrated into a hairbrush comprising a plurality of bristles and are adapted to measure the vibrations of the respective bristles of the hairbrush during the brushing of the hair. Thus, the vibration measurements performed by the vibration sensors during the brushing of the hair are indicative of the vibrations of the bristles of the hairbrush during the brushing. Generally, the vibration sensors can refer to acceleration sensors that measure as vibration of the bristles a respective acceleration of the bristles. For example, a vibration leads to repeated accelerations in different directions such that the acceleration values measured by the vibration sensors as vibration signal are indicative of the vibrations of the bristles.

Thus, in a preferred embodiment, the vibration signal refers to a time sequence of measured acceleration values. However, the vibration sensors can also measure other quantities indicative of the vibration of the bristles, for instance, a velocity or velocity change of the bristles or a position or position change of the bristles. In some embodiments, velocity or position measurements can also be utilized for determining acceleration values of the bristles, for instance, by deriving the acceleration values from the velocity or position signal. Thus, although in the following in preferred embodiments acceleration values are utilized, the vibration signal can also refer to a velocity or position signal from which the acceleration values can be derived.

The hair characteristic determination unit is adapted to determine a hair characteristic based on the vibration signal. The hair characteristic can refer to any characteristic of the hair, for instance, to the type of hair like whether the hair is curled or straight, to a thickness of the hair, whether the hair was artificially coloured, bleached or styled, whether the hair was subject to a physical treatment like heating or UV lighting, etc. Preferably, the determined hair characteristic refers to a hair quality. In this context, the hair quality can be defined as being indicative of a damaging of the hair. For example, hair can be damaged by respective hair treatments or environmental impact on the hair, wherein for such hair damage quality factors can be defined. For example, the amount of a hair damage and thus the quality of the hair can be categorized in two or more categories for which different thresholds for the respective characteristics of the hair can be set. Such categories can be determined by respective experimental measurements with respect to hair with a known quality.

In one embodiment, the hair characteristic determination unit is adapted to determine the hair characteristic based on acceleration values of the bristles provided by the measured vibration signal. In particular, one or more of the time sequence of acceleration values provided by the vibration sensors can be utilized by the hair characteristic determination unit. Generally, which acceleration values of the bristles are utilized can depend on which hair characteristic shall be measured. For example, if a type of hair shall be measured, other acceleration values can be utilized than when a quality of the hair, i.e. the hair damage, shall be measured. For example, a hair characteristic can lead to a specific pattern of the amplitudes of the vibration signal such that in this case as measured acceleration values of the bristles the acceleration values at the maxima and minima of the amplitudes of the vibration signal can be utilized. In other cases, i.e. with respect to other characteristics, average values, absolute maximum values, etc. can be utilized for determining the hair characteristic such that for these other cases other measured acceleration values can be utilized. Moreover, the vibration signal can also be prepared before determining the specific patterns or other signal characteristics. For example, filters can be utilized to remove noise or predetermined frequency ranges from the vibration signal. In particular, the result of the hair characteristic determination can be improved by removing during a signal preparation extreme data points, i.e. data points that fall outside of predetermined maximum and minimum thresholds. Which acceleration values can be utilized can be easily determined by performing a sequence of measurements of the vibrations of the bristles of hair with a respective known characteristic.

In a preferred embodiment, the hair characteristic determination unit is adapted to determine an average value of the acceleration values of the bristles of a brushing stroke and further to determine a deviation value indicative of the deviation of the acceleration values of the bristles of a brushing stroke from the average acceleration value of the bristles of that brushing stroke and to determine the hair characteristic based on the deviation value. In particular, for determining the average value of the acceleration values of the bristles of one brushing stroke, all acceleration values can be utilized that belong to the vibration signal of one brushing stroke. Preferably, the average value is determined for one brushing stroke, however, in some embodiments it may also be advantageous to determine the average value by averaging over more than one brushing stroke. For example, if the measured vibration signals are for some reasons particularly fluctuating, averaging over more than one brushing stroke can allow for a higher accuracy. Based on the such determined average value, the deviation value can then be determined. In particular, a deviation of the acceleration values of the bristles of a brushing stroke from the average acceleration value can be determined. For example, the deviation of all acceleration values provided by the vibration signal from the average acceleration value can be determined. However, also a subset of the acceleration values provided by the vibration signal can be utilized for determining the deviation. The subset can, for instance, refer to an arbitrary subset, for example, to every third acceleration value, or to a selected subset, for instance, to the acceleration values at the maxima and minima of the respective vibration amplitudes of the bristles. The such determined deviations can then be utilized to determine the deviation value. For example, the determined deviations can be averaged and the average can be determined as a deviation value. However, also a maximum deviation can be utilized as deviation value. In particular, the inventors have found that such a deviation value is in particular indicative of the hair quality and thus can be preferably utilized to determine a hair quality.

In another preferred embodiment, the hair characteristic determination unit is adapted to apply a second integration over time to acceleration values of the bristles provided by the vibration measurements and to determine the hair characteristic based on the resulting second integrated signal. A second integration over time refers to integrating the acceleration values, for example, by integrating the vibration signal, over time and then integrating the result of this integration again over time. The resulting second integrated signal can also be regarded as referring to a displacement signal indicative of a displacement of the bristles during the brushing. It has been found by the inventors that based on the hair characteristic the displacement of the bristles during a brushing stroke is different. In particular, for determining a hair quality the respective second integration signal is suitable. Preferably, the characteristic determination unit is adapted to determine for a maximum and/or minimum of the second integrated signal a span width of the second integrated signal and to determine the hair characteristic based on the determined span width. In this context the span width is determined as the difference between the highest/lowest value of the maximum/minimum to the lowest/highest value of a directly previous or following minimum/maximum value. However, the hair characteristic determination unit can also be adapted to utilize alternative or additional characteristics of the vibration signal. For example, the number or widths of amplitudes of the vibration signal can be indicative of respective hair characteristics.

In an embodiment, the apparatus further comprises a velocity signal providing unit for providing a velocity signal generated by a velocity sensor integrated into the hairbrush, wherein the velocity signal is indicative of the brushing velocity, and wherein the hair characteristic determination unit is adapted to determine the hair characteristic further based on the velocity signal. Providing in addition to the vibration signal a velocity signal allows to provide further information. In particular, the vibration signal measured by the vibration sensors can depend on the velocity with which the hair is brushed. Accordingly, the velocity signal can be utilized during the determination of the hair characteristic. For example, if for determining the hair characteristic the vibration signals of more than one brushing stroke shall be utilized, for instance, by averaging respective values of different brushing strokes, the velocity signal can be utilized for weighting the respective vibration signals or to utilize only vibration signals from brushing strokes comprising a velocity lying within a predetermined velocity range. Moreover, for some hair characteristics the characteristics of the vibration signals can depend on the brushing velocity. Thus, also for determining a respective hair characteristic a velocity signal can directly be utilized, for instance, by determining a respective hair characteristic depending on a vibration signal characteristic and a velocity, particularly if the vibration signal characteristic depends on the velocity. Moreover, the hair characteristic determination unit further can be adapted to utilize the velocity signals as controlling signals for controlling a measurement or determination of the hair characteristic. For example, gesture recognition algorithms can be used to derive gestures performed with the hairbrush from the velocity signals. Such gestures, like shaking gestures, can then be associated with a respective controlling of the hair characteristic determination, for example, can then start or stop the measurement, etc.

In an embodiment, the apparatus further comprises a rotation signal providing unit for providing a rotation signal generated by a rotation sensor integrated into the hairbrush, wherein the rotation signal is indicative of a degree of rotation of the hairbrush during the brushing, and wherein the hair characteristic determination unit is adapted to determine the hair characteristic further based on the rotation signal. Generally, the vibration signal can also depend on the degree of rotation of the hairbrush during the brushing, for instance, an angle between the bristles and the brushed hair can have an influence on the vibration of the bristles. Such dependencies can be taken into account during the determination of the hair characteristic based on the rotation signal. Moreover, also the degree of rotation of the hairbrush with respect to the hair can be utilized in a way as described above with respect to the velocity signal. For example, if more than one brushing stroke shall be utilized for determining the hair characteristic, the degree of rotation of the hairbrush during a brushing stroke can be taken into account during averaging processes between the vibration signals of the different brushing strokes. For example, for determining the hair characteristic the hair characteristic determination unit can be adapted to only utilize brushing strokes for which the rotation signal indicates that they lie within a predetermined degree of rotation range or a weighting can be applied to vibration signals from brushing strokes with different degrees of rotation. Moreover, for determining a respective hair characteristic a degree of rotation can directly be utilized, for instance, by determining a respective hair characteristic depending on a vibration signal characteristic and a degree of rotation, particularly if the vibration signal characteristic depends on the degree of rotation.

In an embodiment, the apparatus further comprises an acoustic signal providing unit for providing an acoustic signal generated by an acoustic sensor integrated into the hairbrush, wherein the acoustic signal is indicative of a sound generated while brushing the hair, and wherein the hair characteristic determination unit is adapted to determine the hair characteristic further based on the acoustic signal. The acoustic sensor can be, for instance, a microphone or other sound detecting sensor. In particular, the hair characteristic determination unit can be adapted to evaluate spectral sound variations indicative of hair characteristics of a user. For example the additional usage of the sound signal allows to even more accurately determine hair quality changes, for instance, due to hair treatment with a hair treatment product.

In an embodiment, the apparatus further comprises an image signal providing unit for providing an image signal generated by an image sensor integrated into the hairbrush, wherein the image signal provides an image of the hair and/or scalp of a user while brushing the hair, and wherein the hair characteristic determination unit is adapted to determine the hair characteristic further based on the image signal. The image sensor can be, for instance, a camera or any other light detecting sensor. Preferably the image sensor is configured to detect light ranging from UV-light with a wavelength of 160 nm to infrared light with a wavelength of 20 μm. In particular, the hair characteristic determination unit can be adapted to utilize the image signal to additional or in combination with the vibration signal determine a hair characteristic and/or a scalp characteristic of the user.

In an embodiment, the apparatus further comprises a force signal providing unit for providing a force signal generated by a force sensor integrated into the hairbrush, wherein the force signal is indicative of a force acting between the hairbrush and/or one or more bristles of the hair brush during brushing, and wherein the hair characteristic determination unit is adapted to determine the hair characteristic further based on the force signal. The force sensor can be, for instance, an acceleration sensor, a pressure sensor, or any other sensor that can be utilized to derive a force acting between the hair and the brush and/or one or more bristles. In particular, the hair characteristic determination unit can be adapted to utilize the force signal to additional or in combination with the vibration signal determine a hair characteristic of the user, for example, a change of a air characteristic caused by a hair treatment.

In an embodiment, the apparatus further comprises a humidity signal providing unit for providing a humidity signal generated by a humidity sensor integrated into the hairbrush, wherein the humidity signal is indicative of a humidity of the hair and/or the scalp of a user and/or external air during brushing, and wherein the hair characteristic determination unit is adapted to determine the hair characteristic further based on the humidity signal. The humid sensor can be realized as any sensor that allows to determine a moisture in the environment of the sensor. In particular, the hair characteristic determination unit can be adapted to utilize the humidity signal to additional or in combination with the vibration signal determine a hair characteristic of the user. Since an external humidity can have an effect on hair characteristics measuring the external humidity of the air surrounding the user allows to take the effect of the humidity into account when assessing the hair quality. Moreover, a moisture of the hair or scalp of a user can also influence the hair characteristics, for example, wet hair will have very different combing characteristics than dry hair. Thus, also this effect can be taken into account. In some cases the apparatus can even be adapted to notify a user that an assessment is not possible, due to the hair being to wet or dry based on the measured humidity of the hair and/or scalp.

Generally, all above described additional signals, i.e. the velocity signal, the rotation signal, the humidity signal, the force signal, image signal and acoustic signal, can also be utilized in any combination with the vibration signal for determining a hair characteristic of a user. In particular, all additional sensors can be provided and the hair characteristics determination unit can be adapted to utilize predetermined rules indicating for which cases which signal combination are to be utilized for determining the hair characteristic. For example, if the humidity signal indicates wet hair, the rules can determine that for determining the hair characteristics of wet hair a combination of the vibration signal and the acoustic signal can be utilized to get accurate results, whereas for dry hair additionally the force signal can also be taken into account. In a further aspect of the invention, a hairbrush for accessing hair characteristics while brushing hair of a subject is presented, wherein the hairbrush comprises a) a plurality of bristles, and b) one or more vibration sensors integrated into the hairbrush such that the one or more vibration sensors measure a vibration signal indicative of vibrations of the bristles of the hairbrush.

Generally, the hairbrush can refer to any known construction of a hairbrush comprising a plurality of bristles for brushing a hair of a subject. For example, the bristles can be made of a metal or a synthetic material. Also natural material like wood can be used as basis for the bristles. Moreover, the brush can have different forms and functions. The vibration sensors can be integrated into the hairbrush by any known manner. In particular, the vibration sensors are integrated such that they can measure a vibration signal indicative of the vibrations of the bristles of the hairbrush. Thus, the vibration sensors are integrated such that they have a direct or indirect contact to the bristles of the hairbrush. A direct contact can be provided, for instance, if the vibration sensors are integrated into the hairbrush to be in direct contact with the bristles, for instance, at a measurement surface. An indirect contact in this context refers to a contact in which a further material is provided between the vibration sensors and the bristles, wherein the additional material allows to transfer the vibration of the bristles to the vibration sensors. Generally, a vibration sensor can be integrated such that it mainly measures the vibrations of one bristle. However, a vibration sensor can also be integrated such that it measures the vibrations of a plurality of bristles, for instance, of a predetermined subset of all bristles of the brush. Preferably, at least two vibration sensors are provided and integrated in the hairbrush, however, also only one vibration sensor allows for a suitable measurement accuracy while measuring the vibration signals of the bristles of the hairbrush. In a preferred embodiment, one vibration sensor is integrated into the hairbrush by arranging the vibration sensor at a center of a bristle support structure, for instance, a membrane, such that it measures the vibrations of one or more bristles arranged at or around the center of the bristle support structure.

Preferably, the vibration sensors are integrated into the hairbrush by adhering the one or more vibration sensors to one or more bristles of the hairbrush with a silicon based adhesive. Adhering the vibration sensors to the bristles of the hairbrush with a silicon based adhesive has the advantage that the silicon based adhesive allows for a good transfer of the vibrations of the bristles to the vibration sensors. Moreover, the silicon adhesive can also function as a levelling agent that allows to place the one or more vibration sensors in a constant distance to a supporting structure of the bristles, for instance, a membrane. However, also a thermoplastic glue, preferably, on an ethylene-vinyl acetate basis, can be utilized and provides similar advantages. Furthermore, the above preferred adhesives have the advantages that they provide a good longevity, resistance against water or moisture and UV light and further are not too brittle, such that they are, in particular, suitable for being used in an expected environment of a hairbrush.

The vibration sensors can refer to any kind of sensor that allows to measure a vibration. Preferably, the vibration sensors refer to an acceleration sensor that measures the vibrations of the bristles in form of acceleration values of the bristles. For example, the acceleration sensors can refer to piezo ceramic or quartz vibration sensors. However, the vibration sensor can also refer to a velocity or position sensor, wherein a velocity sensor is in this case adapted to measure the vibrations by measuring the changing velocity of the bristles and a position sensor is adapted to measure the vibrations by measuring the changing position of the bristles during their vibration.

In a preferred embodiment, the hairbrush further comprises a velocity sensor adapted to measure a velocity signal indicative of a velocity of the hairbrush and/or a rotation sensor adapted to measure a rotation signal indicative of a degree of rotation of the hairbrush. Integrating one or more velocity sensors and/or one or more rotation sensors into the hairbrush allows to provide further data on the movement of the hairbrush that can be taken into account when determining a hair characteristic using the hairbrush. Some principles how the additional velocity signal and rotation signal can be utilized when determining a hair characteristic are already given above. Generally, the velocity and/or the rotation sensor can be integrated into the hairbrush in a plurality of ways, for instance, they can be integrated into a handle of the hairbrush or can be attached to a back surface of the hairbrush that does not comprise bristles. The velocity sensor and/or the rotation sensor are preferably provided such that they have a good contact to the hairbrush such that they can generally follow the movements of the hairbrush and thus measure the velocity of the hairbrush and the degree of rotation of the hairbrush. Additionally or alternatively further sensors, in particular, an acoustic sensor adapted to measure a sound generated during brushing, an image sensor adapted to take images of the hair and/or scalp of a user during brushing, a force sensor adapted to measure a force between the hair and the brush and/or one or more bristles of the brush, and/or a humidity sensor adapted for measuring a moisture of the hair, scalp and/or external air during brushing, can be integrated into the brush. Also these sensors can be integrated into the brush in any technical sensible way that allow the sensors to take the respective measurements. For example, an image sensor can be integrated into the handle, the sides or the front of the brush such that the images can be taken during the brushing.

In an embodiment the brush can further comprise a three axis gyroscope, wherein the brush can be configured to be controllable utilizing the signals of the gyroscope. For instance the gyroscope signals can be indicative of gestures of a user made while holding the brush and the brush can comprise a controller configured to control the brush based on these signals.

In an embodiment the hair brush can comprise a user interface for interfacing with the user. The user interface can be realized in any way that allows to provide information to a user. For example, the user interface can be configured to provide haptic feedback, e.g. utilizing vibrations, audio feedback, e.g. utilizing a sound, or visual feedback, e.g. utilizing blinking and/or coloured LEDs. The information provided to the user via the interface can refer to any information relevant for the user when using the brush. For example, the information can be controlling information like indicating if the user is going too fast, indicating the start or stop of a measurement or indicating a successful measurement. The information can also refer to the results of a measurement, for instance, to the determined hair quality.

In a further aspect of the invention, a hairbrush system for accessing hair characteristics while brushing hair of a subject is presented, wherein the system comprises a) a hairbrush as described above, and b) an apparatus as described above.

In an embodiment, the hairbrush system further comprises a user interface, wherein the apparatus is communicatively coupled to the user interface to provide a result of the hair characteristic determination to a user. The user interface can be provided in a plurality of different ways. For instance, the user interface can be provided as part of a software application on a computational device of the user, for instance, a smartphone, a tablet, a laptop, a personal computer, etc. In this case, the apparatus is adapted to be communicatively coupled to the respective computational device. Moreover, the apparatus can itself be part of the respective computational device of the user. However, the user interface can also be realized as being part of the hairbrush. For example, a small display can be integrated into a handle of the hairbrush that allows to show the hair characteristics to a user. In this case, the apparatus is communicatively coupled to the hairbrush such that the result of the hair analysis can be provided to the user interface on the hairbrush. However, the apparatus itself can also be part of the hairbrush, for instance, can be integrated as a dedicated hardware and/or software into the hairbrush, for example, by providing a computational device in a handle of the hairbrush. Moreover, the user interface can also be integrated into a loading and/or general base station utilized for loading or otherwise interacting with the brush, for instance, holding the brush. Generally, the user interface does not necessarily have to refer to a visual user interface that provides visual signals to a user. In particular, the user interface can also refer to a user interface that provides audio signals to a user like speech signals, beeps, melodies, or other signals. Moreover, the user interface can also be a very simple user interface comprising, for instance, only a light like an LED light that allows to provide differently coloured light to the user, wherein the colour of the light provided by the user interface depends on the determined hair characteristic. For example, if the hair quality is determined, such a light can provide a green light if the hair quality is within normal standards, a yellow light if the hair quality deviates from a hair that is normally considered as being healthy, and a red light if the hair is according to normal standards considered to be badly damaged.

In a further aspect of the invention, a method for accessing hair characteristics while brushing hair of a subject is presented, wherein the method comprises a) providing a vibration signal indicative of a result of a vibration measurement performed by one or more vibration sensors integrated into a hairbrush comprising a plurality of bristles, wherein the vibration measurements are performed by the vibration sensors during a brushing of the hair using the hairbrush and are indicative of vibrations of the bristles of the hairbrush during the brushing, and b) determining a hair characteristic based on the vibration signal.

In a further aspect of the invention, a computer program product for accessing hair characteristic is presented, wherein the computer program product comprises program code means for causing the apparatus as described above to execute the method as described above.

It shall be understood that the apparatus as described above, the hairbrush as described above, the system as described above, the method as described above and the computer program product as described above have similar and/or identical preferred embodiments, in particular, as defined in the dependent claims.

It shall be understood that a preferred embodiment of the present invention can also be any combination of the dependent claims or above embodiments with the respective inde-pendent claim.

These and other aspects of the present invention will be apparent from and elucidated with reference to the embodiments described hereinafter.

### BRIEF DESCRIPTION OF THE DRAWINGS

FIG. 1 shows schematically and exemplarily a system for assessing hair characteristics by brushing hair of a subject,

FIG. 2 shows schematically and exemplarily a flowchart of a method for assessing hair characteristics by brushing hair of a subject, and

FIGS. 3a to 5c show experimental results of determining a hair characteristic utilizing the system for assessing hair characteristics.

### DETAILED DESCRIPTION OF THE INVENTION

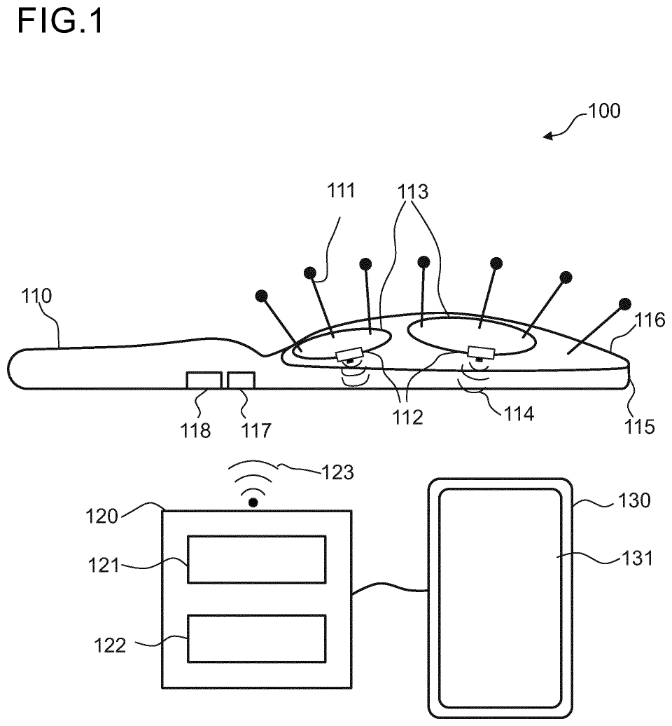

FIG. 1 shows schematically and exemplarily an embodiment of a system 100 for assessing hair characteristics by brushing hair of a subject. The system 100 comprises a hairbrush 110 for brushing hair of a subject and an apparatus 120 for assessing the hair characteristics of the hair being brushed. In a preferred embodiment, the apparatus 120 is in communicative contact to or integrated as part of a hardware and/or software into a computational user device 130, like a smartphone. However, in other embodiments the apparatus 120 can also be a standalone device specifically usable for determining the hair quality. Moreover, the apparatus 120 can also be integrated into the hairbrush 110, for instance, into a handle of the hairbrush 110.

The hairbrush 110 refers to a generally known hairbrush and comprises a standard hairbrush body 115 and a plurality of bristles 111. The bristles 111 can be attached to a flexible part 116 of the hairbrush 110 or can be directly attached to the hairbrush 110. Also the bristles 111 can refer to standard metal or synthetic bristles that are attached to the main body of the hairbrush 110 by any known method. For example, in the embodiment shown in FIG. 1 the bristles 111 are attached via a bristle support structure, here realized as a flexible part 116, e.g. flexible membrane, to the main body 115 of the hairbrush 110. Further, the hairbrush 110 comprises one or more vibration sensors 112 integrated into the hairbrush 110. In the example shown in FIG. 1, two vibration sensors 112 have been integrated into the hairbrush 110. However, also only one sensor can be integrated into the hairbrush 110 or more than two sensors can be integrated into the hairbrush 110. The vibration sensors 112 are integrated into the hairbrush 110 by adhering the vibration sensors to the bristles 111 of the hairbrush 110. Preferably, an adhesive 113 for adhering the vibration sensors 112 to the bristles 111, in particular a silicone based adhesive, is utilized.

However, also other methods for adhering or attaching the vibration sensors to one or more of the bristles 111 can be utilized that allow the vibration sensors 112 to measure the vibrations of the bristles 111. In the example shown in FIG. 1, the utilized adhesive 113 allows a vibration sensor 112 to measure the vibrations of more than one bristle 111, in particular, of all bristles that are connected to the vibration sensor 112 via the adhesive 113. Arranging the vibration sensor 112 such that it can measure the vibrations of more than one bristle 111 allows generally to measure a more accurate vibration signal and allows to reduce exceptional outliers and other abnormalities in the vibration measurement signal. However, as will also be shown below, also integrating the one or more vibration sensors 112 such that each vibration sensor 112 can only measure the vibrations of one bristle 111 allows for a suitable accuracy for determining a hair characteristic. Generally, the vibration sensors 112 can refer to any kind of sensor that allows to measure vibrations of the bristles 111, i.e. that allows to provide a measurement signal that is indicative of the back-and-forth movements of the bristles 111 during a brushing stroke through the hair of a subject. However, preferably, the vibration sensors 112 refer to acceleration sensors that measure an acceleration in the vibration movement of the bristles 111. In other embodiments, the vibration sensors 112 can also be velocity sensors that measure a velocity of the bristles 111 during the brushing of the hair or position sensors that measure a position or a change in position of the bristles 111.

In a preferred but optional embodiment, the hairbrush 110 can further comprise a velocity sensor 117 and/or a rotation sensor 118. The velocity sensor 117 is preferably integrated into the hairbrush 110 such that it can measure an overall velocity of the hairbrush during the brushing. The rotation sensor 118 is preferably integrated into the hairbrush 110 such that it can measure an overall rotation, in particular, a degree of rotation of the hairbrush. Generally, all sensors mentioned above, i.e. the vibration sensors 112, the velocity sensor 117 and the rotation sensor 118, can be adapted to either measure an absolute value of the respective measurement quantity or a relative value and thus a change of the respective measurement quantity. Also further sensors like an acoustic sensor, an image sensor, a humidity sensor, and/or a force sensor can additionally or alternatively be integrated into the hairbrush 110.

Moreover, the above mentioned sensors are generally communicatively coupled to the apparatus 120 as indicated by the wireless communication signals 114 and 123. Generally, the realization of the communicative coupling between the sensors 112, 117, 118 and the apparatus 120 can depend on the respective realization of the apparatus 120. For example, if the apparatus 120 is integrated into a handle of the hairbrush 110, the communicative coupling can be provided by respective signal cables. However, if the apparatus 120 is realized as a standalone device, or is part of a computational device of a user, the communicative coupling can be realized in form of wireless communication signals utilizing any known wireless communication standard. In particular, in this case, a dedicated communication unit, not shown in FIG. 1, can be provided as part of the hairbrush 110 to which the sensors are communicatively coupled, for instance, utilizing communication signal wires, wherein the communication unit is then adapted to forward the measurement signals of the sensors to the apparatus 120 via a respective known wireless communication technique.

The apparatus 120 comprises a vibration measurement providing unit 121 and a hair characteristic determination unit 122. The vibration measurement providing unit 121 is adapted to provide the vibration signals that are indicative of the result of the vibration measurements performed by the one or more vibration sensors 112. For example, the vibration measurement providing unit 121 can be adapted as a receiving unit for directly receiving the vibration signals measured by the vibration sensors 112 and for providing the received vibration signals. However, the vibration signals measured by the vibration sensors 112 can also be provided to a storage unit, for instance, to a cloud storage, wherein in this case the vibration measurement providing unit 121 is adapted to access the respective storage unit and to provide the stored vibration signals to the hair characteristic determination unit 122. Generally, the vibration signal is indicative of the vibrations of the bristles 111 of the hairbrush 110 during the brushing of the hair of a subject.

The hair characteristic determination unit 122 is then adapted to determine a hair characteristic based on the respective vibration signals. It has been found by the inventors that in particular the vibration signals of the bristles 111 of a hairbrush 110 are suitable to determine a plurality of hair characteristics, for instance, a type of the hair, how the hair was treated in the past, and, in particular, a quality of the hair, i.e. whether and to which degree the hair has been damaged. In particular, the hair characteristic determination unit 122 can be adapted to utilize characteristics of the vibration signal that are associated with the respective hair characteristics. For example, as will be shown in the following, characteristics of the signal like a standard deviation from an average signal value or a span width of a second integration of the vibration signal can be directly associated with hair characteristics, in particular with a hair quality and subtleness of the hair.

Moreover, the hair characteristic determination unit 122 can in a preferred embodiment also utilize the results of the velocity measurement provided by the velocity sensor 117 and the results of the rotation measurement provided by the rotation sensor 118 for determining a hair characteristic. In particular, these additional signals can be utilized for further increasing an accuracy of the determination of the hair characteristic. For example, based on the velocity signal provided by the velocity sensor 117, the beginnings and endings of hair strokes can be easily determined and the velocity of the different hair strokes can be compared. This allows to utilize, for instance, only vibration signals coming from brushing strokes performed with the velocity of the hairbrush 110 in a predetermined similarity range. Accordingly, it can be avoided to compare brushing strokes performed with strongly deviating velocities which might lead to respective strongly deviating vibrations of the bristles of the hairbrush. Moreover, also the angle between the hairbrush and the hair with which the brushing is performed can have an influence on the respective vibration of the bristles. Thus, taking the degree of rotation provided by the rotation sensor 118 into account during the hair characteristic determination also allows to only utilize the vibration signals of brushing strokes within a predetermined degree of rotation of the hairbrush 110 that has been shown to be optimal for the determination of the hair characteristic. Also the signals of the other potential additional sensor can be utilized by the hair characteristic determination unit 122 to improve the measurements of the hair characteristics, for example, by utilizing predetermined rules on how and when to combine the respective signals for determining the air characteristics.

The respective result of the hair characteristic determination, i.e. the respective hair characteristic like the respective quality of the hair, can then be provided to a user device 130 and can be displayed, for instance, on display 131. In a preferred embodiment, the result refers to a hair quality score and is presented on the display 131 of the user device 130, for instance, by utilizing a respective app on the user device 130. However, the result can also be provided to a user in other forms, for instance, via a light or a small display provided as part of the hairbrush 110 or via an audio signal like a beep or a voice to a loudspeaker that can be provided to the user device 130 or can also be provided to the hairbrush 110. Moreover, information to the user, like a result, can also be provided to a user via a loading and/or base station of the hairbrush.

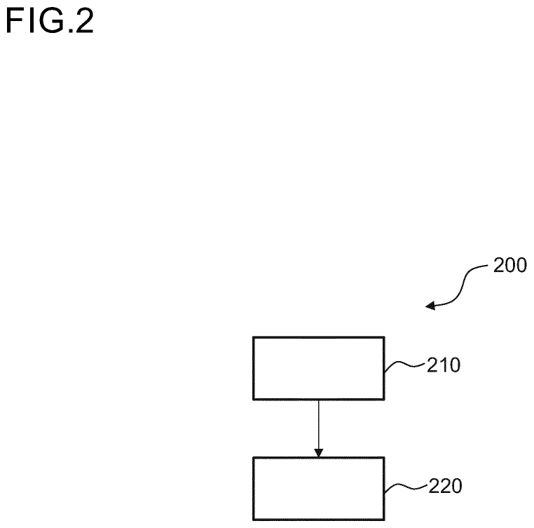

FIG. 2 shows schematically and exemplarily a flowchart of a method for assessing hair characteristics brushing hair of a subject. The method 200 comprises a first step 210 of providing a vibration signal indicative of the result of a vibration measurement performed by one or more vibration sensors integrated into a hairbrush comprising a plurality of bristles. In particular, a hairbrush as shown in FIG. 1 can be utilized for providing the vibration signal in accordance with the above described principles. Further, the method 200 comprises a step 220 of determining a hair characteristic based on the provided vibration signal. The hair characteristic can be determined also in accordance with the respective principles that are described above and the further details that are described in the following.

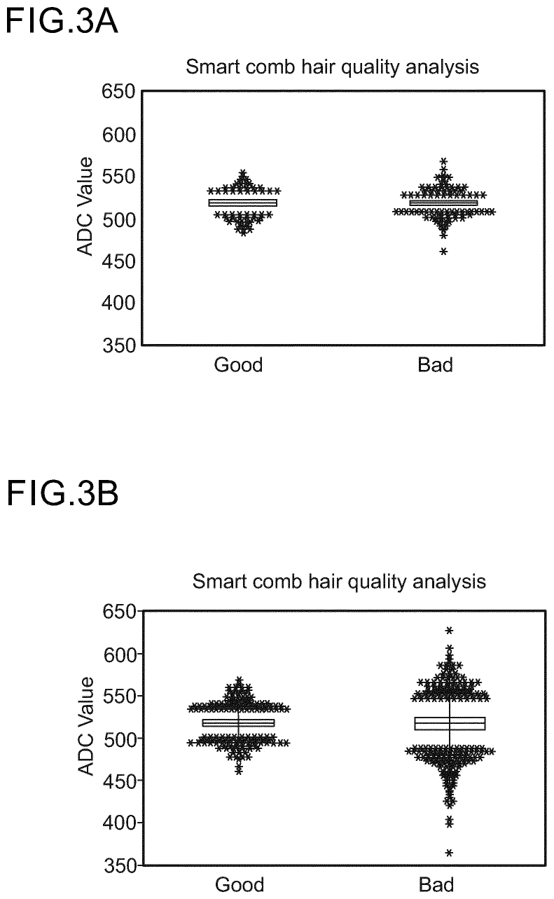

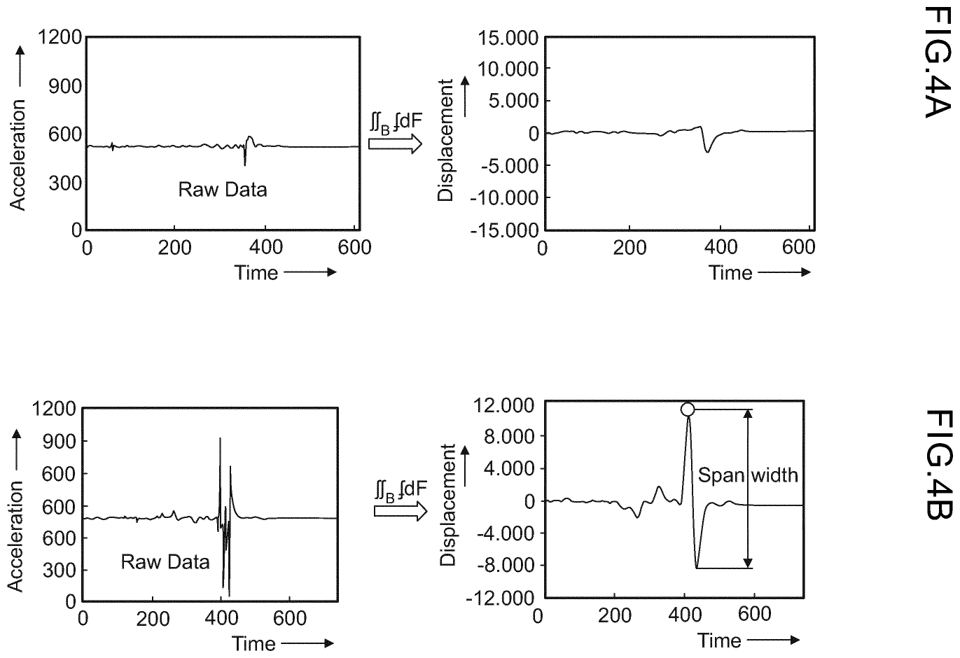

In the following, with respect to FIGS. 3a to 5c, results of a hair characteristic determination will be described in detail with respect to preferred embodiments of the invention. For the following examples, underneath the bottom side of a commercialized hairbrush, vibration sensor has been integrated with a silicone adhesive.

In a first example, utilizing a hairbrush as described above, hair with a known good and bad quality, i.e. according to a respective standard evaluation healthy hair and damaged hair, is combed with the hairbrush as described above. FIG. 3A shows that the vibration signals for good and bad hair can be clearly distinguished. The FIG. 3A shows that the average values of the signals are comparable, but that a differentiation of the two different hair qualities is possible by the deviation of the signal values from the average.

In a further example, shown in FIG. 3B the density of bristles of the hairbrush is increased and the same adhesive is used to connect a plurality of bristles of the hairbrush to the vibration sensor. Although already in the above example, the differentiation was easily possible, FIG. 3A shows that by connecting a plurality of bristles to the vibration sensor the possible differentiation of good and bad hair quality can be increased up to 300%. Thus, the accuracy of a hair quality determination can be significantly increased.

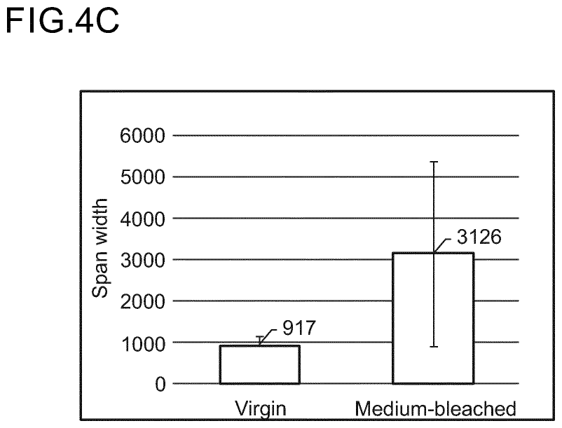

In a further example, differently damaged Caucasian hairs strands are evaluated utilizing a hairbrush as described above. In the experimental setup the hairbrush is fixed horizon-tally in a material testing machine and the hair strands with the known damaging status are pulled through the bristles of the brush using holding clamps at the top and bottom side of the hairbrush. The displacement velocity, i.e. the brushing velocity, is adjusted to 5 cm/s. In this case, the vibrational sensor refers to an acceleration sensor and the acceleration signals of the vibrational sensor are recorded over time. In this case the apparatus is adapted to apply a second integration to the signal. FIG. 4A shows the result of the second integration of the vibration signal for undamaged hair and FIG. 4B for damaged hair, in this case in particular bleached hair. The second integration of the acceleration signal results in displacement values, shown on the right of the FIGS. 4A and 4B that can then be further evaluated for determining the hair quality or other characteristics. For example, as shown in FIG. 4B a span width of the maximum and minimum values of the second integration signal can be determined. As can be derived from FIGS. 4A and 4B a second integrated signal from medium-bleached hair is more distinctive than a second integrated signal from the untreated, i.e. virgin, hair. FIG. 4C shows the significantly different mean values of the span width of five virgin hair strands and of five medium-bleached hair strands both evaluated after the second integration.

Mannequin heads had been washed with 3 different shampoos from a drugstore. Before-hand the shampoos had been evaluated by measuring residual dry combing work respectively suppleness using Caucasian hair strands. FIG. 6 shows a depiction of these two parameters to differentiate the shampoos. The lower the values of these parameters the easier the dry combing respectively the better the smoothness and softness of the hair strands.

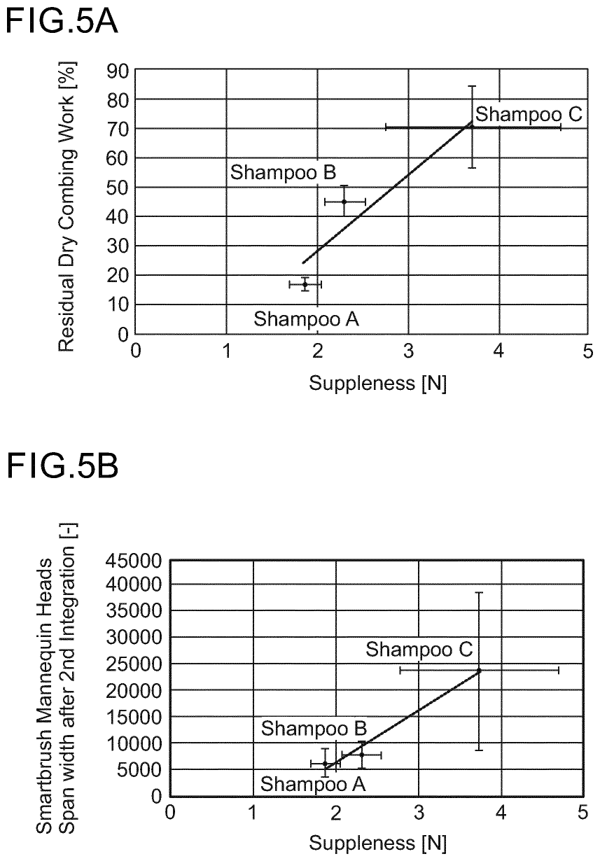

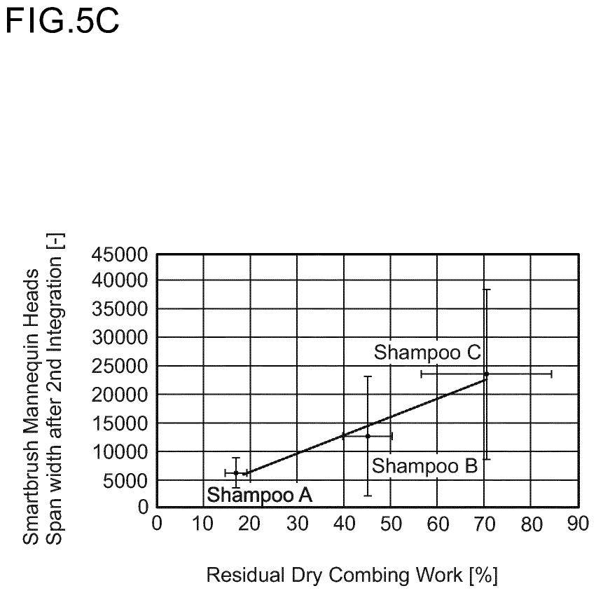

In a further example, three mannequin heads are washed twice with different respective shampoos A, B, C and dried overnight. Each mannequin head associated with a different shampoo is then combed with a hairbrush as described above. In this experiment the hairbrush is used manually with 3 combing strokes at 10 repetitions. Moreover, for each mannequin also a suppleness of the hair is measured. For example, a dry hair strand of the mannequin is pulled through a displacement configuration, e.g. a number of rings alternat-ingly spaced apart, and the force necessary for pulling the hair strand through the displacement is measured. The measured force is then indicative of the suppleness of the hair stand, in particular, the lower the pulling force the higher the suppleness. FIG. 5B show mean span width values after second integration of the acceleration signals, calculated as described above, in relation to a measured suppleness for the three different shampoos. FIG. 5A shows for the same experimental setup the calculated mean span width values after second integration of the acceleration signals in relation to residual dry combing work values for the three shampoos. For measuring the dry combing work values hair strands of the respective mannequin heads can be combed with a predefined configuration of combs that are oppositely arranged and the force can then be measured that is needed for pulling the hair strand through the comb arrangement. From the force the dry combing work can then be derived. Moreover, FIG. 5C shows for all three shampoos the relation between the suppleness and the residual dry combing work values. FIGS. 5A, 5B and 5C show that the characteristics of the vibration signal, here the span width of the second integrated vibration signal, allow for an accurate differentiation between hair with different characteristics, here different suppleness or dry combing work values. Moreover, since the different hair characteristics are caused by different shampoos, the utilization of the vibration signal for determining the hair characteristics allows even to determine the influence of hair care products on the hair of a user.

All the above examples indicate the vibration signals of bristles determined utilizing a hairbrush as described above allow for an accurate determination of a hair characteristic, in particular, of a hair quality. Moreover, vibration signals provided by respectively integrated vibration sensors in a hairbrush are suitable to differentiate between differently damaged hair and different hair treatments which makes it an easy way to use device for hairdressers and consumers for helping with the selection of respective hair care products for an individual user.

The present invention refers to a “smart” hairbrush that utilized a vibration sensor to analyze different hair characteristics, in particular, hair qualities, while brushing. Generally, there is a growing trend for home-use evaluation of hair care products by end-consumers themselves. The evaluation becomes possible by providing “smart” devices, i.e. mainly wireless standalone devices that are able to track individual hair and scalp condition and/or improvement. However, there are more scalp and less hair evaluation devices available on the market. Moreover, there is also a need to get a reliable and efficient device which can be directly and easily used by the end-consumer. The technique of the respective device should be self-explaining and the results of the application should be visible either directly on the device or on a connected cell phone. There are some devices for hair analysis today, which, however, are only designed for usage by hairdressers, i.e. professionals. Other devices for hair evaluation, which are described in literature or other media, cannot be purchased or are only using imaging techniques which is often incomprehensible and inapplicable for the consumer. These devices are in particular costly and the information provided by the devices often inaccurate such that probably no consumer would be prepared to spend money for this kind of information.

In this context, it was found by the inventors that from vibration signals of the bristles of a hairbrush hair characteristic can be accurately and easily derived. Thus, a hairbrush for hair quality analysis using a vibration sensor can be advantageously utilized for hair characteristic analysis. For example, a vibration sensor can be utilized that is adapted to detect vibration from 10 Hz to 15 kHz through a piezo ceramic material base sensor with vibration signal amplification circuit integration. Such a hairbrush together with the respective apparatus, as described in the examples above, can be easily used during daily hair treatment without additional hair expertise.

It is further noted, that generally there are a lot of other approaches for smart hair analysis, like utilizing infrared light, ultraviolet light, visible light respectively imaging, force or sound measurements, but that all these techniques have certain drawbacks. Especially the evaluation of absorption curves from different light sources can be impaired by product residues on hair. Force and sound measurements strongly depend on individual consumer application and hair style. In contrast thereto the inventors have found that utilizing a vibration sensor is a very robust technique which can directly measure the health status of the hair fiber. Healthy hair is very smooth and flexible. Damaging caused by different hair treatment, like bleaching, UV irradiation or straightening by heat or chemicals, or even hair washing results in more brittle hair fibers. Hereby hair proteins are oxidized respectively hair lipids are lost and the natural appearance of the hair is impaired. Thus, the vibration signal analysis as described in this application can be utilized to allow users to avoid these damages and to provide respective indications how such damages can be repaired. The vibration sensor technology is unique and could be integrated in standard hairbrushes.

Other variations to the disclosed embodiments can be understood and effected by those skilled in the art in practicing the claimed invention, from a study of the drawings, the dis-closure, and the appended claims.

For the processes and methods disclosed herein, the operations performed in the processes and methods may be implemented in differing order. Furthermore, the outlined operations are only provided as examples, and some of the operations may be optional, combined into fewer steps and operations, supplemented with further operations, or expanded into additional operations without detracting from the essence of the disclosed embodiments.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Procedures like the providing of vibration signals, the determining of the hair characteristic, etc. performed by one or several units or devices can be performed by any other number of units or devices. These procedures can be implemented as program code means of a computer program and/or as dedicated hardware.

A computer program product may be stored/distributed on a suitable medium, such as an optical storage medium or a solid-state medium, supplied together with or as part of other hardware, but may also be distributed in other forms, such as via the Internet or other wired or wireless telecommunication systems.

Any units described herein may be processing units that are part of a classical computing system. Processing units may include a general-purpose processor and may also include a field programmable gate array (FPGA), an application specific integrated circuit (ASIC), or any other specialized circuit. Any memory may be a physical system memory, which may be volatile, non-volatile, or some combination of the two. The term “memory” may include any computer-readable storage media such as a non-volatile mass storage. If the computing system is distributed, the processing and/or memory capability may be distributed as well. The computing system may include multiple structures as “executable components”. The term “executable component” is a structure well understood in the field of computing as being a structure that can be software, hardware, or a combination thereof. For instance, when implemented in software, one of ordinary skill in the art would under-stand that the structure of an executable component may include software objects, rou-tines, methods, and so forth, that may be executed on the computing system. This may include both an executable component in the heap of a computing system, or on computer-readable storage media. The structure of the executable component may exist on a com-puter-readable medium such that, when interpreted by one or more processors of a computing system, e.g., by a processor thread, the computing system is caused to perform a function. Such structure may be computer readable directly by the processors, for instance, as is the case if the executable component were binary, or it may be structured to be interpretable and/or compiled, for instance, whether in a single stage or in multiple stages, so as to generate such binary that is directly interpretable by the processors. In other in-stances, structures may be hard coded or hard wired logic gates, that are implemented exclusively or near-exclusively in hardware, such as within a field programmable gate array (FPGA), an application specific integrated circuit (ASIC), or any other specialized circuit. Accordingly, the term “executable component” is a term for a structure that is well understood by those of ordinary skill in the art of computing, whether implemented in software, hardware, or a combination. Any embodiments herein are described with reference to acts that are performed by one or more processing units of the computing system. If such acts are implemented in software, one or more processors direct the operation of the computing system in response to having executed computer-executable instructions that constitute an executable component. Computing system may also contain communication channels that allow the computing system to communicate with other computing systems over, for example, network. A “network” is defined as one or more data links that enable the transport of electronic data between computing systems and/or modules and/or other electronic devices. When information is transferred or provided over a network or another communica-tions connection, for example, either hardwired, wireless, or a combination of hardwired or wireless, to a computing system, the computing system properly views the connection as a transmission medium. Transmission media can include a network and/or data links which can be used to carry desired program code means in the form of computer-executable instructions or data structures and which can be accessed by a general-purpose or special-purpose computing system or combinations. While not all computing systems require a user interface, in some embodiments, the computing system includes a user interface system for use in interfacing with a user. User interfaces act as input or output mechanism to users for instance via displays.

Those skilled in the art will appreciate that at least parts of the invention may be practiced in network computing environments with many types of computing system configurations, including, personal computers, desktop computers, laptop computers, message processors, hand-held devices, multi-processor systems, microprocessor-based or programmable consumer electronics, network PCs, minicomputers, mainframe computers, mobile tel-ephones, PDAs, pagers, routers, switches, datacenters, wearables, such as glasses, and the like. The invention may also be practiced in distributed system environments where local and remote computing system, which are linked, for example, either by hardwired data links, wireless data links, or by a combination of hardwired and wireless data links, through a network, both perform tasks. In a distributed system environment, program modules may be located in both local and remote memory storage devices.

Those skilled in the art will also appreciate that at least parts of the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configura-ble computing resources, e.g., networks, servers, storage, applications, and services. The definition of “cloud computing” is not limited to any of the other numerous advantages that can be obtained from such a model when deployed. The computing systems of the figures include various components or functional blocks that may implement the various embodiments disclosed herein as explained. The various components or functional blocks may be implemented on a local computing system or may be implemented on a distributed computing system that includes elements resident in the cloud or that implement aspects of cloud computing. The various components or functional blocks may be implemented as software, hardware, or a combination of software and hardware. The computing systems shown in the figures may include more or less than the components illustrated in the figures and some of the components may be combined as circumstances warrant.

Any reference signs in the claims should not be construed as limiting the scope.

The invention refers to an apparatus for accessing hair characteristic while brushing hair of a subject, wherein the apparatus comprises a vibration measurement providing unit for providing a vibration signal indicative of a result of a vibration measurement performed by one or more vibration sensors integrated into a hairbrush comprising a plurality of bristles. The vibration measurements are performed by the vibration sensors during a brushing of the hair using the hairbrush and are indicative of vibrations of the bristles of the hairbrush during the brushing. A hair characteristic determination unit then determines a hair characteristic based on the vibration signal.

1. An apparatus (120) for accessing a hair characteristic while brushing hair of a subject, wherein the apparatus (120) comprises:
a vibration measurement providing unit (121) for providing a vibration signal indicative of a result of the vibration measurement performed by one or more vibration sensors (112) integrated into a hairbrush (110) comprising a plurality of bristles (111), wherein the vibration measurement is performed by the vibration sensors (112) during a brushing of the hair using the hairbrush (110) and are indicative of vibrations of the plurality of the bristles (111) of the hairbrush (110) during the brushing, and
a hair characteristic determination unit (122) for determining the hair characteristic based on the vibration signal.

2. The apparatus (120) according to claim 1, wherein the determined hair characteristic refers to a hair quality.

3. The apparatus (120) according to claim 1, wherein the hair characteristics determination unit (122) is adapted to determine the hair characteristic based on a measured acceleration values of the bristles (111) provided by the vibration signal.

4. The apparatus (120) according to claim 3, wherein the hair characteristic determination unit (122) is adapted to determine an average value of the acceleration value of the bristles (111) of a brushing stroke and further to determine a deviation value indicative of the deviation of the acceleration values of the bristles (111) of a brushing stroke from the average acceleration value of the bristles (111) of that brushing stroke and to determine the hair characteristic based on the deviation value.

5. The apparatus (120) according to claim 1, wherein the hair characteristic determination unit (122) is adapted to apply a second integration over time to the acceleration value of the bristles provided by the vibration measurement and to determine the hair characteristic based on the resulting second integrated signal.

6. The apparatus (120) according to claim 5, wherein the characteristic determination unit (122) is adapted to determine for a maximum and/or minimum of the second integrated signal a span width of the second integrated signal and to determine the hair characteristic based on the determined span width.

7. The apparatus (120) according to claim 1, wherein the apparatus (120) further comprises a velocity signal providing unit for providing a velocity signal generated by a velocity sensor (117) integrated into the hairbrush (110), wherein the velocity signal is indicative for the brushing velocity, and wherein the hair characteristic determination unit (122) is adapted to determine the hair characteristics further based on the velocity signal.

8. The apparatus (120) according to claim 1, wherein the apparatus (120) further comprises a rotation signal providing unit for providing a rotation signal generated by a rotation sensor (118) integrated into the hairbrush (110), wherein the rotation signal is indicative of a degree of rotation of the hairbrush (110) during the brushing, and wherein the hair characteristic determination unit (122) is adapted to determine the hair characteristic further based on the rotation signal.

9. A hairbrush (110) for accessing a hair characteristic while brushing hair of a subject, wherein the hairbrush (110) comprises:
a plurality of bristles (111), and
one or more vibration sensors (112) integrated into the hairbrush (110) such that the one or more vibration sensors (112) measure a vibration signal indicative of vibrations of the bristles (111) of the hairbrush (110).

10. The hairbrush (110) according to claim 9, wherein the vibration sensors (112) are integrated into the hairbrush (110) by adhering the one or more vibration sensors (112) to one or more bristles (111) of the hairbrush (110) with a silicon based adhesive (113).

11. The hairbrush (110) according to claim 9, wherein the hairbrush (110) further comprises a velocity sensor (117) adapted to measure a velocity signal indicative of a velocity of the hairbrush (110) and/or a rotation sensor (118) adapted to measure a rotation signal indicative of a degree of rotation of the hairbrush (110).

12. A hairbrush system (100) for accessing a hair characteristic while brushing hair of a subject, wherein the system (100) comprises:
a hairbrush (110) comprising a plurality of bristles (111), and
an apparatus (120) according to claim 1.

13. The hairbrush system (100) according to claim 12, wherein the hairbrush system (100) further comprises a user interface, wherein the apparatus (120) is communicatively coupled to the user interface to provide a result of the hair characteristic determination to a user.

14. A method (200) for accessing hair characteristic while brushing hair of a subject, wherein the method (200) comprises:
providing (210) a vibration signal indicative of a result of a vibration measurement performed by one or more vibration sensors (112) integrated into a hairbrush (110) comprising a plurality of bristles (111), wherein the vibration measurements are performed by the vibration sensors (112) during a brushing of the hair using the hairbrush (110) and are indicative of vibrations of the bristles (111) of the hairbrush (110) during the brushing, and
determining (220) a hair characteristic based on the vibration signal.

15. A computer program product for accessing a hair characteristic, wherein the computer program product comprises program code for causing an apparatus (120) to execute the method according to claim 14.
